(19) United States
(12) Patent Application Publication
(10) Pub. No.: US 2025/0249227 A1
(43) Pub. Date: Aug. 7, 2025

(54) MICRO-PROJECTION MASSAGE HEAD AND MASSAGER

(71) Applicant: SUZHOU NASHENG MICROELECTRONICS CO., LTD., Suzhou (CN)

(72) Inventor: Bai Xu, Suzhou (CN)

(21) Appl. No.: 19/186,678

(22) Filed: Apr. 23, 2025

Related U.S. Application Data
(63) Continuation-in-part of application No. PCT/CN2023/124549, filed on Oct. 13, 2023.

(30) Foreign Application Priority Data
Oct. 24, 2022 (CN) 202211303849.7

(51) Int. Cl. A61M 37/00

(52) U.S. Cl. CPC A61M 37/0015; A61M 2037/0023; A61M 2037/0061

(57) ABSTRACT

The present disclosure relates to the field of microneedle transdermal enhancement technology, in particular to a micro-projection massage head and a massager, wherein the micro-projection massage head includes a massage nanochip, the micro-projections are provided on the massage nanochip, and the height of the micro-projection does not exceed 60 micrometers; the massager includes a vibrating mechanism and a micro-projection massage head; during operation, the vibration mechanism can drive the micro-projection massage head to vibrate, and the working vibration frequency of the micro-projection massage head is not less than 1000 times per minute. According to the present disclosure, the technical problems such as discomfort and easily causing damage to subcutaneous tissue during the application of the existing microneedle transdermal enhancement technology can be solved, and the improvement effect of the effective ingredients on the skin can be revealed in time.

### CROSS-REFERENCE TO RELATED APPLICATIONS

The present disclosure is a Continuation-In-Part application of PCT Application No. PCT/CN2023/124549 filed on Oct. 13, 2023, which claims the priority to the Chinese patent application with the filling No. 202211303849.7 filed with the Chinese Patent Office on Oct. 24, 2022, and entitled “MICRO-PROJECTION MASSAGE HEAD AND MASSAGER”, the contents of which are incorporated herein by reference in entirety.

### TECHNICAL FIELD

The present disclosure relates to the field of microneedle transdermal enhancement technology, in particular to a micro-projection massage head and a massager.

### BACKGROUND ART

Human skin includes epidermis, dermis and subcutaneous tissue from outside to inside, wherein the epidermis further includes the outermost stratum corneum, as well as the stratum lucidum, stratum granulosum, stratum spinosum, and stratum basale beneath the stratum corneum. The stratum corneum is composed of several to dozens of layers of flattened anucleate corneocytes, which exhibit strong resistance to factors such as acids, alkalis, and friction, and possess protective function as a barrier. When the stratum corneum is too thin, the barrier function of skin will be impaired, leading to skin problems such as allergies and water-oil imbalance. A normal stratum corneum has an effective barrier protective function, which is capable of preventing harmful components such as bacteria and dust from entering the skin, but at the same time, it also hinders the absorption for effective ingredients, resulting in poor transdermal absorption effect, thus affecting the skin care and repair.

For this reason, the microneedle transdermal enhancement technology has been developed within the industry to improve the transdermal absorption efficacy. According to the technology, a delivery channel is formed on the stratum corneum via microneedles, which facilitates the effective ingredients to enter the skin from the delivery channel, thus improving the transdermal absorption efficacy. In order to ensure favorable absorption efficacy, it is generally believed in the industry that the deeper the delivery channel enters the skin, the better the transdermal absorption efficacy is; when the delivery channel runs through the stratum corneum, the transdermal absorption efficacy is better. Given that the thickness of the human stratum corneum ranges between 10-25 micrometers, the height of microneedles in the products on the market is mostly 100 micrometers or above, aiming at penetrating the stratum corneum of skin. Relevant research and technologies can be referenced in Chinese patent “MICRONEEDLE DEVICES AND METHODS (Patent No. CN103429222B)” and Chinese patent application “METALLIC MICRONEEDLES (Publication No. CN104936648A)”, and other literature. Microneedles within this height range can effectively unclog the stratum corneum, so that the effective ingredients can quickly reach the depths of the skin, thereby significantly improving the transdermal absorption efficacy. Clinical studies have further demonstrated that the microneedle transdermal enhancement technology of this size can thicken stratum corneum and gradually restore barrier protective function.

Based on the barrier protective function of the stratum corneum and the development of the research of industry, it is generally recognized in the industry that when the height of microneedles is relatively small, microneedles cannot form an effective delivery channel on the stratum corneum, and is even more unable to penetrate the stratum corneum, and then the effective ingredients cannot quickly enter the skin through the delivery channel, so it is easy to infer that microneedles with relatively small height cannot play an effective role in transdermal enhancement and absorption.

In the prior art, the height of microneedles is mostly selected to be 100 micrometers or above. In the actual application process, the following problems exist.
1. When microneedles within this height range are adopted to perform the transdermal enhancement, the effect is not obvious in a short time, which makes users misunderstand that it cannot produce the effect of transdermal enhancement, thus significantly hindering the popularization of the microneedle transdermal enhancement technology.
2. The microneedles within this height range penetrate deep into the stratum corneum, and even through the stratum corneum, which are prone to cause discomfort during the care process, thus resulting in poor user experience.
3. Due to individual variations, users with relatively thinner stratum corneum of the skin have the risks of subcutaneous tissue damage when adopting microneedles within this height range to perform the transdermal enhancement.

The above problems lead to the failure of effective large-scale popularization of the microneedle transdermal enhancement technology.

### SUMMARY

In order to solve the technical problems of the existing microneedle transdermal enhancement technology, such as long waiting time for visible effects and discomfort during use, a micro-projection massage head is provided in the present disclosure. The micro-projection massage head includes a massage nanochip, wherein micro-projections are provided on the massage nanochip, and the height of the micro-projection does not exceed 60 micrometers.

The micro-projection massage head can be composed of a massage nanochip and a massage handle, the size of the massage nanochip can be from millimeter level to centimeter level or even larger, and the micro-projections are arranged on the surface of the massage nanochip in array. Moreover, the micro-projection massage head can include only massage nanochip. In the present technical solution, the height of the micro-projection does not exceed 60 micrometers. When the micro-projection massage head is adopted to treat the skin surface, the micro-projections only form the relatively shallow delivery channels on the stratum corneum, and the delivery channels do not penetrate the stratum corneum. Therefore, no obvious discomfort is generated during the process of treating the skin surface, thus effectively improving the usage experience. More importantly, due to the small height of the micro-projections and the shallow delivery channels formed, the effective ingredients can be kept in the relatively shallow position of the stratum corneum after entering the skin, so that the improvement effect of the effective ingredients on the skin can be visually reflected in a short period of time, which has the effect of rapid and obvious appearance of the efficacy. In addition, since the effective ingredients are kept only at a relatively shallow position of the stratum corneum when they initially enter the skin, the effective ingredients have little irritation to the skin, so that a wider variety of effective ingredients can be adopted in conjunction with the micro-projection massage head of the present disclosure.

In addition, it is verified by experiments that the effective ingredients kept in the relatively shallow position of the stratum corneum can be fully absorbed by the skin, thus achieving the care effect of traditional microneedles (the height of microneedles is 100 micrometers or above). That is to say, the micro-projection massage head in the present disclosure can promote the full absorption of the effective ingredients and, at the same time, make the improvement effect of the effective ingredients on the skin appear visually in a short period of time, and can alleviate the discomfort of users and reduce the risk of subcutaneous tissue damage.

Preferably, the height of the micro-projection is not greater than the width of the micro-projection.

Preferably, the height of the micro-projection is greater than the width of the micro-projection.

Preferably, the height of the micro-projections does not exceed 45 micrometers.

Preferably, the height of the micro-projections does not exceed 25 micrometers.

Preferably, the height of the micro-projections is less than 10 micrometers. Through the above configuration, the delivery channel formed by the micro-projections is only located in the shallow layer of the stratum corneum, so that the effective ingredients remain in the shallow layer of the stratum corneum. After the micro-projection massage head is adopted to care for the skin surface, the effective ingredients can be visually presented on the skin surface, thus achieving the technical effect of “immediate appearance of care effect”.

In the preferred technical solution, the height of the micro-projections is smaller than the thickness of the stratum corneum, so even if the contact pressure of the micro-projection massage head on the skin surface is relatively large, the micro-projections can be effectively prevented from penetrating through the stratum corneum, thus ensuring that the user has a good experience.

Preferably, the massage nanochip is fabricated from one or more of the following materials: metal materials, polymer materials, monocrystalline silicon materials, or plastic materials.

In addition, a massager is further provided by the present disclosure. The massager includes a vibrating mechanism and a micro-projection massage head according to any one of the above technical solutions. During operation, the vibration mechanism can drive the micro-projection massage head to vibrate, and the working vibration frequency of the micro-projection massage head is not less than 1000 times per minute. In the present technical solution, the massager is able to form dense delivery channels on the skin surface in a short time, and the effective ingredients remain in the shallow layer of the stratum corneum after passing through the delivery channels and immediately appear to the naked eye. Through the above configuration, when the massager drives the high-frequency vibrating micro-projection massage head to move slowly on the skin surface, the improvement effect of the effective ingredients on the skin can be immediately and visibly presented, thus effectively proving the effect of transdermal enhancement and absorption of the massager, so as to promote the popularization of the microneedle transdermal enhancement technology.

Preferably, the working vibration frequency of the micro-projection massage head is not less than 3000 times per minute.

Preferably, during operation, the micro-projection massage head performs the unclogging treatment on the skin surface by contacting with the skin surface, so as to facilitate the transdermal enhancement and absorption for effective ingredients.

In some embodiments, the effective ingredient is in a liquid state, a solid state or a semi-solid state. Because the delivery channels are only formed in the shallow layer of the stratum corneum by the micro-projections provided in the present disclosure, the effective ingredients only remain in the shallow layer of the stratum corneum in a short time after entering the skin, so that the effective ingredients have little irritation to the skin, thus making the application safer and more stable. Accordingly, when the present disclosure is adopted to care the skin surface, the types and forms of the effective ingredients are not overly restricted, so that a wide range of application scenarios and scopes can be achieved.

In some embodiments, the skin surface includes periocular skin.

### BRIEF DESCRIPTION OF THE DRAWINGS

Preferred embodiments of the present disclosure will be described with reference to the accompanying drawings, in which:

FIG. 1 is a schematic structural diagram of the massager according to the present disclosure.

FIG. 2 is a schematic structural diagram of a micro-projection massage head and a connecting member according to the present disclosure.

FIG. 3 is a schematic structural diagram of the massage nanochip of the present disclosure according to Example 1.

FIG. 4 is a schematic structural diagram of the massage nanochip of the present disclosure according to Example 2.

FIG. 5 is a graph of the change of diffusion amount of effective ingredients over time in the skin surface absorption effect experiment.

FIG. 6 is a graph of the change of transepidermal water loss over time in the stratum corneum barrier recovery experiment.

### LIST OF REFERENCE NUMERALS

A. massager; 1. instrument body; 10. mounting port; 11. vibration mechanism; 110. driving end; 12. battery; 2. connecting member; 21. first flange; 22. second flange; 23. spring; 3. micro-projection massage head; 31. massage nanochip; 310. base; 311. micro-projection; 32. massage handle; 321. bearing plate; 322. massage push rod; 33. third flange.

### DETAILED DESCRIPTION OF EMBODIMENTS

Preferred embodiments of the present disclosure will be described below with reference to the accompanying drawings. It should be understood by those skilled in the art that these embodiments are only adopted to explain the technical principles of the present disclosure, and are not intended to limit the protection scope of the present disclosure.

It should be noted that in the description of the present disclosure, the terms of direction or positional relationship indicated by the terms “up”, “down”, “inside” and “outside”, and the like indicate directions or positional relationships based on those shown in the accompanying drawings, which is only for the convenience of description, and does not indicate or imply that the device or element must have a specific orientation, be constructed and operated in a specific orientation, and therefore are not to be understood as limitations of the present disclosure. Furthermore, the terms “first” and “second” are only adopted for descriptive purposes and cannot be understood as indicating or implying relative importance.

In addition, it should be noted that in the description of the present disclosure, unless otherwise specified and limited, the terms “assembly”, “provide” and “connect” should be broadly understood, for example, it can be fixed connection, detachable connection or integrated connection; it can be directly connected, or be indirectly connected through an intermediate medium, further can be connected inside two elements. For those skilled in the art, the specific meanings of the above terms in the present disclosure can be understood according to specific circumstances.

The stratum corneum of the skin serves a barrier protective function, which makes it difficult for in vitro substances including effective ingredients to be absorbed by the skin. The microneedle transdermal enhancement technology treats and massages the skin surface with microneedles to create a delivery channel on the skin surface. Subsequently, the effective ingredients can quickly penetrate through the delivery channel and be absorbed by the skin. Considering the barrier function of the stratum corneum of the skin, it is generally recognized by those skilled in the art that the delivery channel needs to extend into the skin as deeply as possible, and it is better that the delivery channel can penetrate the stratum corneum, only the delivery channel of such depth can allow the effective ingredients to pass the barrier of stratum corneum, thereby improving the absorption effect of the effective ingredients. Since the stratum corneum has a certain thickness, the height of microneedles of the current microneedle transdermal enhancement instrument is mostly designed to be above 100 micrometers, so as to ensure that the delivery channels formed by microneedles can penetrate the stratum corneum, thus ensuring the absorption effect.

It has been verified by experiments that the existing microneedles with the specifications of 100 micrometers or above can indeed effectively promote the absorption of effective ingredients, but have problems such as poor user experience and subcutaneous tissue damage in the practical application process. More importantly, after skincare is performed with microneedle transdermal enhancement, approximately 6 weeks are required before the improvement effect of the skin can be obviously seen, so that the actual effect of the microneedle transdermal enhancement technology is suspected by the users, which seriously affects the further popularization and the service to the public of this technology.

The present disclosure is developed in the process of long-term research on the microneedle transdermal enhancement technology and the process of serving users. When the newly designed micro-projection massage head is adopted with a massager, the improvement effect of effective ingredients on skin can be quickly revealed, which greatly reduces the user's doubts about the microneedle transdermal enhancement technology, greatly improves the sales volume of products, and facilitates the further popularization of the microneedle transdermal enhancement technology.

The technical solution of the present disclosure will be further explained with examples and drawings.

### Example 1

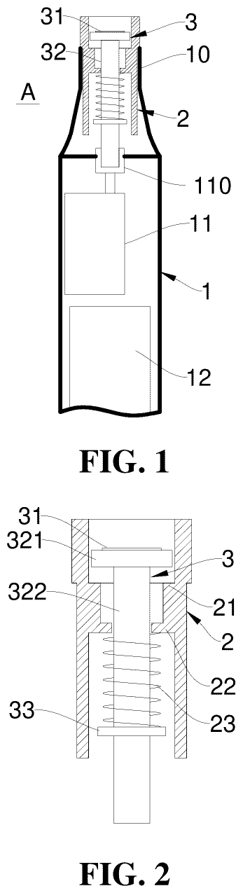

FIG. 1 is a schematic structural diagram of the massager according to the present disclosure. As shown in FIG. 1, the massager A includes an instrument body 1, a connecting member 2 and a micro-projection massage head 3, wherein the micro-projection massage head 3 is detachably assembled on the instrument body 1 through the connecting member 2. The micro-projection massage head 3 or the micro-projection massage head 3 together with the connecting member 2 is adopted as disposable consumables to be matched with the instrument body 1.

As shown in FIG. 1, the end part of the instrument body 1 is provided with a mounting port 10, wherein the mounting port 10 can be threadedly connected to the connecting member 2. Optionally, the outer peripheral wall of the connecting member 2 is provided with an external thread, the inner peripheral wall of the mounting port 10 is provided with an internal thread matched with the external thread, and the connecting member 2 can be partially inserted into the interior of the mounting port 10 and be threadedly connected with it. It is easy to understand that the connecting member 2 can further be partially sleeved on the exterior part of the mounting port 10 and be threadedly connected with it. In addition to the means of threaded connection, the mounting port 10 can further be connected to the connecting member 2 through a snap fit, a plug fit, or by other suitable means.

As shown in FIG. 1, a vibration mechanism 11 is arranged inside the instrument body 1, wherein the vibration mechanism 11 can be powered by a battery 12 arranged inside the instrument body 1. The battery 12 can be a non-detachable built-in battery panel, or can be a replaceable disposable battery or a rechargeable battery. It is easy to conceive that the vibration mechanism 11 can further be connected to an external power supply through a power cord to work.

As shown in FIG. 1, the vibration mechanism 11 is equipped with a driving end 110, wherein the driving end 110 can drive the micro-projection massage head 3 to vibrate, and the working vibration frequency of the micro-projection massage head 3 is not less than 1000 times per minute. In order to improve the care effect of the massager A, the working vibration frequency is preferably 3000 times per minute. It is easy to conceive that the working vibration frequency can further be adjusted to 1500 times per minute, 2000 times per minute or other suitable values according to the skin quality and the needs of users.

FIG. 2 is a schematic structural diagram of a micro-projection massage head and a connecting member according to the present disclosure. As shown in FIG. 2, in some examples, the connecting member 2 is of a hollow cylinder, wherein the outer peripheral wall of the cylinder is provided with an external thread (not shown in the figure) matched with the mounting port 10. The inner peripheral wall of the cylinder is provided with a first flange 21, wherein the first flange 21 is configured to limit the moving range of the micro-projection massage head 3. As shown in FIG. 2, a second flange 22 is further provided on the inner peripheral wall of the cylinder. Optionally, the inner diameter of the second flange 22 is smaller than that of the first flange 21. The second flange 22 is configured to cooperate with the spring 23 to reset the micro-projection massage head 3.

As shown in FIG. 1 and FIG. 2, in the present Example 1, the micro-projection massage head 3 is composed of a massage nanochip 31 and a massage handle 32, wherein the massage handle 32 includes a massage push rod 322 extending along the axis of the cylinder of the connecting member 2, and the outer diameter of the massage push rod 322 is smaller than the inner diameter of the second flange 22. The lower end of the massage push rod 322 passes through the connecting member 2 and then is connected with the driving end 110 of the vibration mechanism 11. Driven by the driving end 110, the massage push rod 322 can freely reciprocate inside the connecting member 2. As shown in FIG. 2, a bearing plate 321 is arranged at the upper end of the massage push rod 322, wherein the outer diameter of the bearing plate 321 is larger than that of the first flange 21, and the first flange 21 can limit the moving range of the massage handle 32 when the bearing plate 321 reciprocates with the massage push rod 322.

In order to improve the rebound effect of the massage handle 32, as shown in FIG. 2, the massage push rod 322 is provided with a third flange 33, wherein the third flange 33 and the second flange 22 are matched to abut against both ends of the spring 23. When the massage handle 32 moves upward to the maximum position, the compressed spring 23 can push the massage handle 32 to move downward to reset, thus improving the contact tightness between the massage handle 32 and the driving end 110.

As shown in FIG. 1 and FIG. 2, the massage nanochip 31 is arranged on the bearing plate 321, and the specific means of fixing thereof can be adhesive adhesion, or can further be fixed connection through snap fit, interference fit or other suitable means.

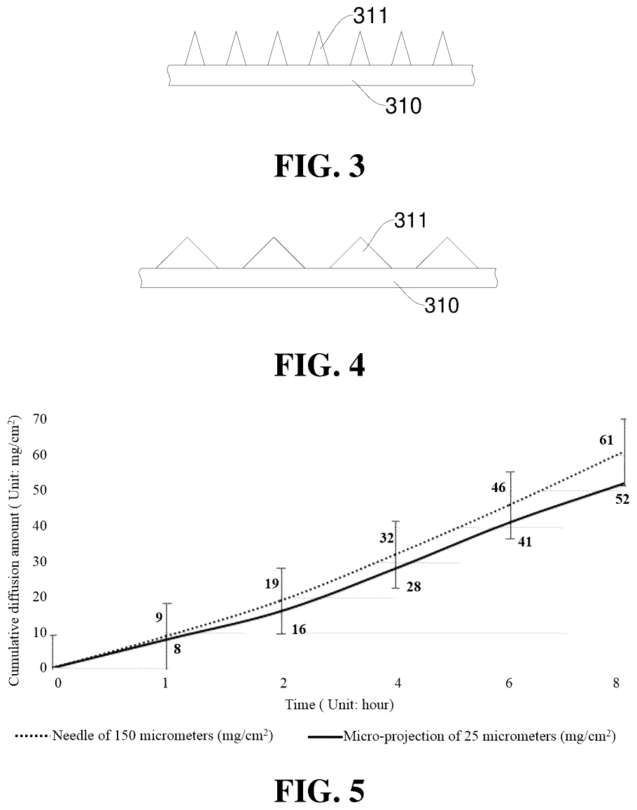

FIG. 3 is a schematic structural diagram of the massage nanochip of the present disclosure according to Example 1. As shown in FIG. 3, the massage nanochip 31 is provided with a base 310, and micro-projections 311 are arranged on the surface of the base 310. As shown in FIG. 3, the micro-projections 311 are arranged in an array on a base 310. It is easy to understand that a plurality of micro-projections 311 are provided, or only one micro-projection 311 is provided. The material of the massage nanochip 31 can be one or more of metal material, polymer material, monocrystalline silicon material and plastic material.

As shown in FIG. 3, in the present Example 1, the height of the micro-projection 311 is greater than the width of the micro-projection 311. It should be emphasized that in the present Example 1, the height of the micro-projections 311 does not exceed 60 micrometers. Optionally, the height of the micro-projections 311 is 60 micrometers, 45 micrometers, 25 micrometers, 10 micrometers, or 9 micrometers, etc. The height of the micro-projections 311 in the present Example 1 is obviously smaller than that of the microneedles selected in the existing microneedle transdermal enhancement technology, and the selection of this size specification breaks the existing cognition in this technical field.

During operation, the vibrating mechanism 11 drives the micro-projection massage head 3 to vibrate to and fro along the axis of the cylinder of the connecting member 2. When the massage handle 32 moves upward to the maximum position, the micro-projection 311 is higher than the connecting member 2 to ensure effective contact with the skin surface.

### Example 2

FIG. 4 is a schematic structural diagram of the massage nanochip of the present disclosure according to Example 2. As shown in FIG. 4, different from Example 1, in the present Example 2, the height of the micro-projection 311 is not greater than the width of the micro-projection 311.

The technical solutions of the present disclosure will be further introduced in combination with the specific application method.

The operation method includes the following steps:
cleaning the skin surface, in which, optionally, the skin surface is the surface of the periocular skin, the cheek skin, or the forehead shin;
coating or spraying effective ingredients on the skin surface, wherein the effective ingredients can be liquid state, solid state or semi-solid state, and optionally, the effective ingredients are brightening essence, whitening essence, sunscreen cream, skin cream, etc.;
starting the massager A, wherein the vibration mechanism 11 drives the micro-projection massage head 3 to vibrate to and fro along the axis of the connecting member 2, with the working vibration frequency more than 1000 times per minute;
adjusting the working vibration frequency as required, wherein, optionally, the working vibration frequency is 3000 times per minute, 3500 times per minute, 4000 times per minute, 4500 times per minute, 5000 times per minute, etc.;
facing and contacting the micro-projection massage head 3 with the skin surface; and
performing unclogging treatment on the skin surface by sliding along the skin surface and pressing/lifting techniques perpendicular to the skin surface.

Taking the effective ingredients as whitening essence as an example, through experimental verification and user experience feedback, through the above operation method, the skin that has been massaged (treated by unclogging) is significantly better in color and smoothness than the skin that has not been massaged (treated by unclogging), and wrinkles can be seen to reduce with naked eyes during the process that the micro-projection massage head 3 slides along the skin surface or presses/lifts perpendicular to the skin surface.

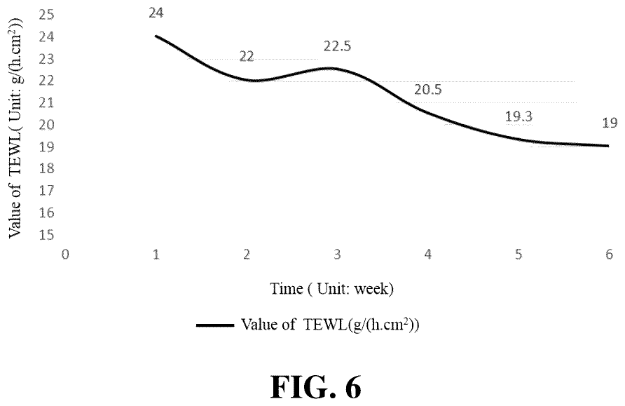

In addition, the effects of the enhancement instrument of the present disclosure in promoting the absorption of effective ingredients and restoring the stratum corneum barrier are further illustrated through experiments. FIG. 5 is a graph of the change of diffusion amount of effective ingredients over time in the skin surface absorption effect experiment; and FIG. 6 is a graph of the change of transepidermal water loss over time in the stratum corneum barrier recovery experiment.

### Skin Surface Absorption Effect Experiment

Methods: The absorption effect of effective ingredients on skin after being treated by unclogging through different massagers is measured by Franz cell method.

Specifically, at room temperature, two adjacent areas of the same kind of skin surface are selected as test points.

A massage nanochip with micro-projections of 25 micrometers is selected to cooperate with the massager provided by the present disclosure to treat one area by unclogging for 20 seconds, wherein the vibration frequency is 3000 times per minute.

At the same time, a nano wafer with a microneedle height of 150 micrometers is selected to cooperate with the massager provided by the present disclosure to treat another area by unclogging for 20 seconds, wherein the vibration frequency is 3000 times per minute, which serves as a comparative experiment.

After the unclogging treatment is completed, the effective ingredients with the same amount and concentration are applied to the two areas respectively.

The cumulative diffusion amounts of the two areas at 0.5 h, 1 h, 2 h, 4 h, 6 h and 8 h are measured by vertical transdermal diffusion instrument respectively. The statistics of experimental data are shown in FIG. 5.

Through the analysis of the above experimental data, it can be seen that the cumulative absorption difference of effective ingredients on the skin surface after being treated in two ways is kept within 15% (as shown in FIG. 5, the cumulative diffusion amount corresponding to the micro-projections of 25 micrometers is always within the 15% error limit of the cumulative diffusion amount corresponding to the needle of 150 micrometers). The experiment shows that the effective ingredients can also be absorbed efficiently by the skin after the skin is treated by unclogging through the massager cooperating with the massage nanochip (in which the height of the micro-projection is less than 60 micrometers) provided by the present disclosure.

Stratum corneum Barrier Recovery Experiment

When the stratum corneum of skin becomes thicker, the barrier protective function thereof becomes better, and the value of transepidermal water loss (TEWL value) decreases. By tracking and measuring the situation of the transepidermal water loss of the skin surface after being treated by unclogging through the massager, the recovery situation of the stratum corneum on the skin surface after being treated by unclogging can be understood. According to the present disclosure, the transepidermal water loss of the skin surface after being treated by unclogging is tracked and measured by the transepidermal water loss tester, and the data is statistically recorded (see FIG. 6). From the data chart, it can be found that within six weeks after the skin surface is treated by unclogging, the value of transepidermal water loss gradually decreases, which reflects that the barrier layer on the skin surface is gradually recovering, and the gradual recovery of the barrier protection function can also be interpreted as the thickening of the skin barrier. Through the analysis of the experimental data in FIG. 6, it can be known that after the skin is treated by unclogging through the massager provided by the present disclosure, the stratum corneum can be stimulated, so that the stratum corneum becomes thicker and the barrier function can be restored.

To sum up, when adopting the micro-projection massage head of the present disclosure to care for skin, not only the absorption of effective ingredients can be promoted effectively, and the stratum corneum can be stimulated to thicken, so as to restore the barrier of the skin, but also the discomfort can be effectively reduced or even eliminated, so that the risk of subcutaneous tissue damage can be avoided. More importantly, it has been experimentally verified that by adopting the massager of the present disclosure to care for skin, the improvement effect of the effective ingredients on the skin can be revealed in time, thus better highlighting the effect of the microneedle transdermal enhancement technology, so as to promote the further popularization of the technology.

So far, the technical solution of the present disclosure has been described in combination with the preferred embodiments shown in the accompanying drawings, but it is easy for those skilled in the art to understand that the protection scope of the present disclosure is obviously not limited to these specific embodiments. Without deviating from the principle of the present disclosure, those skilled in the art can make equivalent changes or substitutions on relevant technical features, and the technical solutions after these changes or substitutions will fall within the protection scope of the present disclosure.

1. A micro-projection massage head, comprising a massage nanochip, wherein a micro-projection is provided on the massage nanochip, and a height of the micro-projection does not exceed 60 micrometers.

2. The micro-projection massage head according to claim 1, wherein the height of the micro-projection is not greater than a width of the micro-projection.

3. The micro-projection massage head according to claim 1, wherein the height of the micro-projection is greater than a width of the micro-projection.

4. The micro-projection massage head according to claim 1, wherein the height of the micro-projections does not exceed 45 micrometers.

5. The micro-projection massage head according to claim 1, wherein the height of the micro-projections does not exceed 25 micrometers.

6. The micro-projection massage head according to claim 1, wherein the height of the micro-projections is less than 10 micrometers.

7. The micro-projection massage head according to claim 1, wherein the massage nanochip is fabricated from one or more of following materials: metal materials, polymer materials, monocrystalline silicon materials, or plastic materials.

8. A massager, comprising:
a vibrating mechanism, and
the micro-projection massage head according to claim 1, wherein
during operation, the vibration mechanism can drive the micro-projection massage head to vibrate, and a working vibration frequency of the micro-projection massage head is not less than 1000 times per minute.

9. The massager according to claim 8, wherein the working vibration frequency of the micro-projection massage head is not less than 3000 times per minute.

10. The massager according to claim 8, wherein during operation, the micro-projection massage head performs an unclogging treatment on a skin surface by contacting with the skin surface, so as to facilitate a transdermal enhancement and absorption of an effective ingredient.

11. The massager according to claim 10, wherein the effective ingredient is in a liquid state, a solid state or a semi-solid state.

12. The massager according to claim 10, wherein the skin surface comprises a periocular skin.

13. The massager according to claim 8, wherein the height of the micro-projection is not greater than a width of the micro-projection.

14. The massager according to claim 8, wherein the height of the micro-projection is greater than a width of the micro-projection.

15. The massager according to claim 8, wherein the height of the micro-projections does not exceed 45 micrometers.

16. The massager according to claim 8, wherein the height of the micro-projections does not exceed 25 micrometers.

17. The massager according to claim 8, wherein the height of the micro-projections is less than 10 micrometers.

18. The massager according to claim 8, wherein the massage nanochip is fabricated from one or more of the following materials: metal materials, polymer materials, monocrystalline silicon materials, or plastic materials.

19. The massager according to claim 9, wherein during operation, the micro-projection massage head performs an unclogging treatment on a skin surface by contacting with the skin surface, so as to facilitate a transdermal enhancement and absorption of an effective ingredient.
